SEQUENCE LISTING (24 sequences)

SEQ ID NO: 1 - DNA, 4107 bp, Homo sapiens
  CDS (54)..(2294)
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 2 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 3 - DNA, 1963 bp, Homo sapiens
  CDS (201)..(1367)
gtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaac

SEQ ID NO: 4 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 5 - DNA, 1931 bp, Homo sapiens
  CDS (257)..(1312)
cgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagaccgactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaacaaaaaaaaaaaaaaaaaaaaaaa

SEQ ID NO: 6 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 7 - DNA, 1927 bp, Homo sapiens
  CDS (103)..(1299)
ggcgccgggggcgggggtgggaggcggaggcggggccggggcgccgcgggcggggcgccgggggcggggcgagtccggaggactcctcggactgcgcggaacatggcgttctggggttggcgcgccgcggcagccctccggctgtggggccgggtagttgaacgggtcgaggccgggggaggcgtggggccgtttcaggcctgcggctgtcggctggtgcttggcggcagggacgatgtgagtgcggggctgagaggcagccatggggcccgcggtgagcccttggacccggcgcgccccttgcagaggcctcccagacccgaggtgcccagggcattccggaggcagccgagggcagcagctcccagtttcttcttttcgagtattaaaggtggaagaaggtccatatctttttctgtgggtgcttcaagtgttgttggaagtggaggcagcagtgacaaggggaagctttccctgcaggatgtagctgagctgattcgggccagagcctgccagagggtggtggtcatggtgggggccggcatcagcacacccagtggcattccagacttcagatcgccggggagtggcctgtacagcaacctccagcagtacgatctcccgtaccccgaggccatttttgaactcccattcttctttcacaaccccaagccctttttcactttggccaaggagctgtaccctggaaactacaagcccaacgtcactcactactttctccggctgcttcatgacaaggggctgcttctgcggctctacacgcagaacatcgatgggcttgagagagtgtcgggcatccctgcctcaaagctggttgaagctcatggaacctttgcctctgccacctgcacagtctgccaaagacccttcccaggggaggacattcgggctgacgtgatggcagacagggttccccgctgcccggtctgcaccggcgttgtgaagcccgacattgtgttctttggggagccgctgccccagaggttcttgctgcatgtggttgatttccccatggcagatctgctgctcatccttgggacctccctggaggtggagccttttgccagcttgaccgaggccgtgcggagctcagttccccgactgctcatcaaccgggacttggtggggcccttggcttggcatcctcgcagcagggacgtggcccagctgggggacgtggttcacggcgtggaaagcctagtggagcttctgggctggacagaagagatgcgggaccttgtgcagcgggaaactgggaagcttgatggaccagacaaataggatgatggctgcccccacacaataaatggtaacataggagacatccacatcccaattctgacaagacctcatgcctgaagacagcttgggcaggtgaaaccagaatatgtgaactgagtggacacccgaggctgccactggaatgtcttctcaggccatgagctgcagtgactggtagggctgtgtttacagtcagggccaccccgtcacatatacaaaggagctgcctgcctgtttgctgtgttgaactcttcactctgctgaagctcctaatggaaaaagctttcttctgactgtgaccctcttgaactgaatcagaccaactggaatcccagaccgagtctgctttctgtgcctagttgaacggcaagctcggcatctgttggttacaagatccagacttgggccgagcggtccccagccctcttcatgttccgaagtgtagtcttgaggccctggtgccgcacttctagcatgttggtctcctttagtggggctatttttaatgagagaaaatctgttctttccagcatgaaatacatttagtctcctcaaagggaaaaaaaaaaaaaaaaaaaaaaaaaaaaaaaaaaaaaaaaaaaaaaaaaaaaaaaaaaaaaaaaaa

SEQ ID NO: 8 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 9 - DNA, 1174 bp, Homo sapiens
  CDS (21)..(962)
gtccgtagagctgtgagagaatgaagatgagctttgcgttgactttcaggtcagcaaaaggccgttggatcgcaaaccccagccagccgtgctcgaaagcctccattgggttatttgtgccagcaagtcctcctctggaccctgagaaggtcaaagagttacagcgcttcatcaccctttccaagagactccttgtgatgactggggcaggaatctccaccgaatcggggataccagactacaggtcagaaaaagtggggctttatgcccgcactgaccgcaggcccatccagcatggtgattttgtccggagtgccccaatccgccagcggtactgggcgagaaacttcgtaggctggcctcaattctcctcccaccagcctaaccctgcacactgggctttgagcacctgggagaaactcggaaagctgtactggttggtgacccaaaatgtggatgctttgcacaccaaggcggggagtcggcgcctgacagagctccacggatgcatggacagggtcctgtgcttggattgtggggaacagactccccggggggtgctgcaagagcgtttccaagtcctgaaccccacctggagtgctgaggcccatggcctggctcctgatggtgacgtctttctctcagaggagcaagtccggagctttcaggtcccaacctgcgttcaatgtggaggccatctgaaaccagatgtcgttttcttcggggacacagtgaaccctgacaaggttgattttgtgcacaagcgtgtaaaagaagccgactccctcttggtggtgggatcatccttgcaggtatactctggttacaggtttatcctcactgcctgggagaagaagctcccgattgcaatactgaacattgggcccacacggtcggatgacttggcgtgtctgaaactgaattctcgttgtggagagttgctgcctttgatagacccatgctgaccacagcctgatattccagaacctggaacagggactttcacttgaatcttgctgctaaatgtaaatgccttctcaaatgacagattccagttcccattcaacagagtagggtgcactgacaaagtatagaaggttctaggtatcttaatgtgtggatattcttaattaaaactcattttttttaaataaaaaattgttcagctttaaaa

SEQ ID NO: 10 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 11 - DNA, 2350 bp, Homo sapiens
  CDS (219)..(1115)
attcgggggcgcgagctgccccagtaaatggaaatgttttctaacatataaaaacctacagaagaagaaaataattttctggatcaaattagaagtctgtattatattgatgtctccagattcaaatatattagaaagcagccgtggagacaaccatcttcattttgggcgaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcagtcatttgatctccatctcatctctaattattataaagaattaaaacaagtcatcattgtagaaaagcaagaaaatgcagatagagaaaaagaagaaaataaaactggagtatttccacaacccaagtttagagttggcccccacctcccatgccatggactgagcagcaggggcccagcatcccttggatatggtggctgtgtcttcatgtgaaagaaactgaacttggtggtttttcctgccagttcaggagagattcttggcatgtaatatatatcactgctcaagtcaagcctcctaaaaccacagacctgtttcagctgctacttcagccaaaattcttcagcttcatattgtcttgaaaacctatgattgtctctaacaaacaggctacttgctagttagaaattcttatcaatttggcaagctacttatcaaccagactgaccacaagaactgtcatctcatcaatgaaggagtaactgatcaatgaagccagcaatgcttttttcttggcatcatcaaagctgacatttagaagagatgctggtgatagtcatctcatcctactcaatttttcaaaggcagaaaccaaccctggagcaattgagaggactgtttaaacacagagcttaacaatggcagaattgtatatctcgtgcttaacagattttggttgaactttaccctaggtcaggggtcagcaaactactgcctgtgggccaaatttgcccaccacctgtatctgtaaataaggtttcattggaacacagctgtggccatatgtttgtatattgtgtgtggctgcttttgcattaggatgacagaggtgaatagttgcaacagagactggctggtctgcaaagcctaaaatatgtcctgtgtggccctttacagaaaaagttttctaacccctgctctaggttacggagaaaaaaaaatggaataatgttctctgctacttttaacctgattttctttgtacctaaataggcagctagaatgctgcctatattttaataaggatttggatctcacaagacaccttaggcctacacaagttgttcagattctttgccccagttctaatctagtgacaaaggcatagaattctcctcccacaggaatgtatttctattttcaaggtgttaattagttccagttttggttttgtcgttttccccatgtccgatgcttatattggatgatttctgataaacctgactattccaataaaccctaggcatttttgaatttaaaaaaaaaaaaaaaaaa

SEQ ID NO: 12 - protein, 299 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFSHLISISSLIIIKN

SEQ ID NO: 13 - DNA, 1670 bp, Homo sapiens
  CDS (297)..(1226)
ccggagcgcggtcgggacacagcgcctctaggagaaagcctggaaggcgctccgggggtacccagagctcttagcgggccggcagcatgtgcggggcccaagtaaatggaaatgttttctaacatataaaaacctacagaagaagaaaataattttctggatcaaattagaagtctgtattatattgatgtctccagattcaaatatattagaaagcagccgtggagacaaccatcttcattttgggagaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttcttaagtgtcctggggaagaaagaaattacagtatatctaagaactaggccacacgcagaggagaaatggtcttatgggtggtgagctgagtactgaacaatctaaaaatagcctctgattccctcgctggaatccaacctgttgataagtgatgggggtttagaagtagcaaagagcacccacattcaaaagtcacagaactggaaagttaattcatattatttggtttgaactgaaacgtgaggtatctttgatgtgtatggttggttattgggagggaaaaattttgtaaattagattgtctaaaaaaaatagttattctgattatatttttgttatctgggcaaagtagaagtcaaggggtaaaaaccctactattctgatttttgcacaagttttagtggaaaataaaatcacactctacagtaaaaaaaaaaaaaaaaaa

SEQ ID NO: 14 - protein, 310 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 15 - DNA, 1638 bp, Homo sapiens
  CDS (61)..(1125)
gcttccggcggaagcggcctcaacaagggaaactttattgttcccgtggggcagtcgaggatgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatggagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctgaccagggtgcttggggagggtggggctttttgtagaaactgtggattctttttctctcgtggtctcactttgttacttgtttctgtccccgggagcctcagggctctgagagctgtgctccaggccaggggttacacctgccctccgtggtccctccctgggctccaggggcctctggtgcggttccgggaagaagccacaccccagaggtgacagctgagcccctgccacaccccagcctctgacttgctgtgttgtccagaggtgaggctgggccctccctggtctccagcttaaacaggagtgaactccctctgtccccagggcctcccttctgggccccctacagcccaccctacccctcctccatgggccctgcaggaggggagacccaccttgaagtgggggatcagtagaggcttgcactgcctttggggctggagggagacgtgggtccaccaggcttctggaaaagtcctcaatgcaataaaaacaatttctttcttgcaaa

SEQ ID NO: 16 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMEHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 17 - DNA, 1718 bp, Homo sapiens
  CDS (34)..(1233)
gcggaagcggaagagcaggtctccaggggagcgatggcagccgggggtctgagccgctccgagcgcaaagcggcggagcgggtccggaggttgcgggaggagcagcagagggagcgcctccgccaggtgtcgcgcatcctgaggaaggcggcggcggagcgcagcgccgaggagggccggctgctggccgagagcgcggacctggtaacggagctgcagggccggagccggcggcgcgagggcctgaagcggcggcaggaggaggtgtgcgacgacccggaggagctgcgggggaaggtccgggagctggccagcgccgtccggaacgccaaatacttggtcgtctacacaggcgcgggaatcagcacggcagcgtctatcccagactaccggggccctaatggagtgtggacactgcttcagaaagggagaagcgttagtgctgccgacctgagcgaggccgagccaaccctcacccacatgagcatcacccgtctgcatgagcagaagctggtgcagcatgtggtgtctcagaactgtgacgggctccacctgaggagtgggctgccgcgcacggccatctccgagctccacgggaacatgtacattgaagtctgtacctcctgcgttcccaacagggagtacgtgcgggtgttcgatgtgacggagcgcactgccctccacagacaccagacaggccggacctgccacaagtgtgggacccagctgcgggacaccattgtgcactttggggagagggggacgttggggcagcctctgaactgggaagcggcgaccgaggctgccagcagagcagacaccatcctgtgtctagggtccagcctgaaggttctaaagaagtacccacgcctctggtgcatgaccaagccccctagccggcggccgaagctttacatcgtgaacctgcagtggaccccgaaggatgactgggctgccctgaagctacatgggaagtgtgatgacgtcatgcggctcctcatggccgagctgggcttggagatccccgcctatagcaggtggcaggatcccattttctcactggcgactcccctgcgtgctggtgaagaaggcagccacagtcggaagtcgctgtgcagaagcagagaggaggccccgcctggggaccggggtgcaccgcttagctcggcccccatcctagggggctggtttggcaggggctgcacaaaacgcacaaaaaggaagaaagtgacgtaatcacgtgctcgatgaagaacagttggcactttgcagatggccagtgtcacggtgaaggctgggttgcccccacgggtctagggagaacgaactctttggggatgacattttcaccgtgacatttttagccatttgtccttgaggaagccccttgcactgctgcggttgtaccctgatacggcctggccatcgaggacacctgcccatccggcctctgtgtcaagaggtggcagccgcacctttctgtgagaacggaactcgggttatttcagccccggcctgcagagtggaagcgcccagcggcctttcctcgctcaccaggccagtctcagggcctcaccgtatttctactactacttaatgaaaaagtgtgaactttatagaatcctctctgtactggatgtgcggcagaggggtggctccgagcctcggctctatgcagacctttttatttctattaaacgtttctgcactggcaaaaa

SEQ ID NO: 18 - protein, 400 aa, Homo sapiens
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 19 - DNA, 2156 bp, Homo sapiens
  CDS (71)..(1897)
gcgcatgcgtggattgtcgtcttctgtccaagttggtcgcttccctgcgccaaagtgagcagtagccaacatgtcagggtgggagtcatattacaaaaccgagggcgatgaagaagcagaggaagaacaagaagagaaccttgaagcaagtggagactataaatattcaggaagagatagtttgatttttttggttgatgcctccaaggctatgtttgaatctcagagtgaagatgagttgacaccttttgacatgagcatccagtgtatccaaagtgtgtacatcagtaagatcataagcagtgatcgagatctcttggctgtggtgttctatggtaccgagaaagacaaaaattcagtgaattttaaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcacctgaagaaacctgggggctttgacatatccttgttctacagagatatcatcagcatagcagaggatgaggacctcagggttcactttgaggaatccagcaagctagaagacctgttgcggaaggttcgcgccaaggagaccaggaagcgagcactcagcaggttaaagctgaagctcaacaaagatatagtgatctctgtgggcatttataatctggtccagaaggctctcaagcctcctccaataaagctctatcgggaaacaaatgaaccagtgaaaaccaagacccggacctttaatacaagtacaggcggtttgcttctgcctagcgataccaagaggtctcagatctatgggagtcgtcagattatactggagaaagaggaaacagaagagctaaaacggtttgatgatccaggtttgatgctcatgggtttcaagccgttggtactgctgaagaaacaccattacctgaggccctccctgttcgtgtacccagaggagtcgctggtgattgggagctcaaccctgttcagtgctctgctcatcaagtgtctggagaaggaggttgcagcattgtgcagatacacaccccgcaggaacatccctccttattttgtggctttggtgccacaggaagaagagttggatgaccagaaaattcaggtgactcctccaggcttccagctggtctttttaccctttgctgatgataaaaggaagatgccctttactgaaaaaatcatggcaactccagagcaggtgggcaagatgaaggctatcgttgagaagcttcgcttcacatacagaagtgacagctttgagaaccccgtgctgcagcagcacttcaggaacctggaggccttggccttggatttgatggagccggaacaagcagtggacctgacattgcccaaggttgaagcaatgaataaaagactgggctccttggtggatgagtttaaggagcttgtttacccaccagattacaatcctgaagggaaagttaccaagagaaaacacgataatgaaggttctggaagcaaaaggcccaaggtggagtattcagaagaggagctgaagacccacatcagcaagggtacgctgggcaagttcactgtgcccatgctgaaagaggcctgccgggcttacgggctgaagagtgggctgaagaagcaggagctgctggaagccctcaccaagcacttccaggactgaccagaggccgcgcgtccagctgcccttccgcagtgtggccaggctgcctggccttgtcctcagccagttaaaatgtgtttctcctgagctaggaagagtctacccgacataagtcgagggactttatgtttttgaggctttctgttgccatggtgatggtgtagccctcccactttgctgttccttactttactgcctgaataaagagccctaagtttgtactatatactgttaaaaaaaaaaaaaaaaaaaaaaa

SEQ ID NO: 20 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 21 - DNA, 2376 bp, Homo sapiens
  CDS (28)..(1500)
cgcgcggcccctgtcctccggcccgagatgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcgagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcattaggctttatgactgggtgtgtgttgtgtgtatgtaatacataatgtttattgtacagatgtgtggggtttgtgttttatgatacattacagccaaattatttgttggtttatggacatactgccctttcattttttttcttttccagtgtttaggtgatctcaaattaggaaatgcatttaaccatgtaaaagatgagtgctaaagtaagctttttagggccctttgccaataggtagtcattcaatctggtattgatcttttcacaaataacagaactgagaaacttttatatataactgatgatcacataaaacagatttgcataaaattaccatgattgctttatgtttatatttaacttgtatttttgtacaaacaagattgtgtaagatatatttgaagtttcagtgatttaacagtctttccaacttttcatgatttttatgagcacagactttcaagaaaatacttgaaaataaattacattgccttttgtccattaatcagcaaataaaacatggccttaacaaagttgtttgtgttattgtacaatttgaaaattatgtcgggacataccctatagaattactaaccttactgccccttgtagaatatgtattaatcattctacattaaagaaaataatggttcttactggaatgtctaggcactgtacagttattatatatcttggttgttgtattgtaccagtgaaatgccaaatttgaaaggcctgtactgcaattttatatgtcagagattgcctgtggctctaatatgcacctcaagattttaaggagataatgtttttagagagaatttctgcttccactatagaatatatacataaatgtaaaatacttacaaaagtgg

SEQ ID NO: 22 - protein, 491 aa, Homo sapiens [sequence omitted]

SEQ ID NO: 23 - DNA, 1240 bp, Homo sapiens
  CDS (38)..(1141)
gagctcgcagcgcgcggcccctgtcctccggcccgagatgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcgagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagaggtatgtgttttatattaaaagtttcaataaggcatttcttataattaagtttgtttatgtttgataaagaacacaatataaatacaaaaaaaaaaaaaaaaaaaaaaaaaaaaa

SEQ ID NO: 24 - protein, 368 aa, Homo sapiens
MNPAAEAEFNILLATDSYKVTHYKQYPPNTSKVYSYFECREKKTENSKLRKVKYEETVFYGLQYILNKYLKGKVVTKEKIQEAKDVYKEHFQDDVFNEKGWNYILEKYDGHLPIEIKAVPEGFVIPRGNVLFTVENTDPECYWLTNWIETILVQSWYPITVATNSREQKKILAKYLLETSGNLDGLEYKLHDFGYRGVSSQETAGIGASAHLVNFKGTDTVAGLALIKKYYGTKDPVPGYSVPAAEHSTITAWGKDHEKDAFEHIVTQFSSVPVSVVSDSYDIYNACEKIWGEDLRHLIVSRSTQAPLIIRPDSGNPLDTVLKVLEILGKKFPVTENSKGYKLLPPYLRVIQGDGVDINTLQEVCVLY

(19) United States
(12) Patent Application Publication
(10) Pub. No.: US 2009/0117543 A1
(43) Pub. Date: May 7, 2009

(54) METHODS AND COMPOSITIONS FOR INDUCING SIRTUINS

(75) Inventor: David A. Sinclair, Chestnut Hill, MA (US)

Correspondence Address: WOLF GREENFIELD & SACKS, P.C., 600 ATLANTIC AVENUE, BOSTON, MA, 02210-2206, US

(73) Assignee: PRESIDENT AND FELLOWS OF HARVARD COLLEGE, Cambridge, MA (US)

(21) Appl. No.: 11/579,382

(22) PCT Filed: May 4, 2005
(86) PCT No.: PCT/US2005/015715

§ 371 Date: Dec. 29, 2008

Related U.S. Application Data
(60) Provisional application No. 60/568,150, filed on May 4, 2004.

(51) Int. Cl. C12Q 1/68; C07K 14/00; C12Q 1/34; C12N 5/00; C12N 1/00

(52) U.S. Cl. 435/6; 530/350; 435/18; 435/375; 435/243

(57) ABSTRACT

Provided herein are compositions comprising agents that increase the level of expression of a sirtuin gene. Also provided are methods for purifying and isolating a factor from serum that stimulates the expression of a sirtuin gene. Diagnostic methods for determining whether a subject is calorically restricted or resistant to stress are also provided.

### CROSS-REFERENCE TO RELATED APPLICATIONS

This application claims the benefit of U.S. Provisional Application No. 60/568,150, filed May 4, 2004, the content of which is specifically incorporated by reference herein.

### BACKGROUND

In mammals, caloric restriction (CR) delays the onset of numerous age-associated diseases including cancer, atherosclerosis and diabetes, and can significantly increase median and maximum lifespan (1). The molecular mechanisms underlying this effect are not known. In the budding yeast Saccharomyces cerevisiae, CR extends lifespan by increasing the activity of Sir2 (2-5), a member of the conserved sirtuin family of NAD⁺-dependent deacetylases (6). In yeast and C. elegans, lifespan is extended by extra copies of the SIR2/Sir-2.1 gene (7, 8) or by small molecule sirtuin agonists (9). In mammals, it is becoming increasingly apparent that SIRT1 is a key regulator of cell defenses and cell survival in response to stress (10-14).

In response to damage or stress, cells may attempt to repair or defend themselves, but if unsuccessful, can undergo programmed cell death called apoptosis. Numerous studies show that aging is associated with increased rates of stress-induced apoptosis (15, 16) and the cumulative effects of cell loss have been implicated in various diseases including neurodegeneration, retinal degeneration, cardiovascular disease and frailty (16-20). Consistent with this, rodents subjected to CR or long-lived genetic mutants such as the p66^{sch} knockout mouse are typically less prone to stress-induced apoptosis (15, 19, 21, 22). A critical step in initiating stress-induced apoptosis is the relocalization of the Bax protein from the cytoplasm to the outer mitochondrial membrane and the subsequent release of cytochrome c. Recent work has identified an important regulatory step in this pathway. Under normal conditions, Bax is rendered inactive by its tight association with the Ku70 protein (23, 24). In response to acute cell damage or stress, two critical lysines in Ku70 (K539, K542) become acetylated by the acetyltransferases CBP and PCAF and the Ku70-Bax interaction is disrupted, allowing Bax to localize to mitochondria (24).

### SUMMARY

Provided herein are compositions, e.g., comprising a fraction of serum of a calorically-restricted organism, wherein the fraction of serum induces the expression of a sirtuin, e.g., SIRT1. A composition may be enriched in an agent that induces the expression of a sirtuin, identified by a method comprising, e.g., (i) obtaining one or more fractions of serum from a calorically-restricted organism; (ii) contacting the one or more fractions with a cell; (iii) determining the level of expression of a sirtuin in the cell, wherein a higher level of expression of the sirtuin in the cell contacted with a fraction relative to a cell that was not contacted with the fraction indicates that the fraction contains an agent that induces the expression of a sirtuin; and (iv) obtaining one or more subfractions of a fraction that contains an agent that induces the expression of a sirtuin and repeating steps (ii) and (iii). The method may further comprise repeating step (iv).

Also provided herein are methods comprising contacting serum from a calorically-restricted organism with a cell and determining the level of expression of a sirtuin in the cell. A method may be used for determining the presence of an agent that induces the expression of a sirtuin in serum or a fraction thereof from a calorically-restricted organism, and may comprise: (i) contacting a cell with a composition comprising serum or a fraction thereof from a calorically-restricted organism; and (ii) determining the level of expression of a sirtuin in the cell; wherein a higher level of sirtuin expression in the cell contacted with the composition comprising serum or a fraction thereof from a calorically-restricted organism relative to a composition that does not comprise serum from a calorically-restricted organism or relative to another fraction of serum from the calorically-restricted organism indicates that the serum or fraction thereof from the calorically-restricted organism comprises an agent that induces the expression of a sirtuin. A method may also be used for enriching a composition in an agent that induces the expression of a sirtuin, and may comprise: (i) obtaining one or more fractions of serum from a calorically-restricted organism; (ii) contacting the one or more fractions with a cell; (iii) determining the level of expression of a sirtuin in the cell, wherein a higher level of expression of the sirtuin in the cell contacted with a first fraction relative to a cell that was not contacted with the fraction or contacted with a second fraction indicates that the first fraction contains an agent that induces the expression of a sirtuin; and (iv) obtaining one, or more subfractions of the fraction that contains an agent that induces the expression of a sirtuin and repeating steps (ii) and (iii), to thereby enrich a composition in an agent that induces the expression of a sirtuin. A method may further comprise repeating step (iv). A method may also be used for identifying a factor in serum from a calorically-restricted organism that induces the expression of a sirtuin, and may comprise: (i) obtaining one or more fractions of serum from a calorically-restricted organism; (ii) contacting the one or more fractions with a cell; (iii) determining the level of expression of a sirtuin in the cell, wherein a higher level of expression of the sirtuin in the cell contacted with a first fraction relative to a cell that was not contacted with the fraction or contacted with a second fraction indicates that the first fraction contains an agent that induces the expression of a sirtuin; (iv) obtaining one or more subfractions of the fraction that contains an agent that induces the expression of a sirtuin and repeating steps (ii) and (iii); and (v) repeating steps (i)-(iv) for a number of times sufficient to identify the factor that induces the expression of a sirtuin.

Other methods that are provided include methods for increasing the expression of a sirtuin in a cell and/or reducing the susceptibility of a cell to apoptosis. A method may comprise contacting the cell with serum or a fraction thereof from a calorically-restricted organism. A method may further comprise determining the level of expression of a sirtuin in the cell. The fraction may be a composition enriched in an agent present in serum from a calorically-restricted organism or an isolated or purified factor therefrom. Methods for reducing the susceptibility of a cell to apoptosis may further comprise increasing the protein or activity level of Ku70 in the cell.

Methods also include those for identifying an agent that increases the expression of a sirtuin gene. A method may comprise (i) contacting a cell comprising a reporter gene operably linked to a transcriptional control region of a sirtuin gene with serum or a fraction thereof or factor purified, enriched or isolated therefrom, from a calorically-restricted organism; and (ii) determining the level of expression of the reporter gene, wherein a higher level of expression of the reporter gene in the cell contacted with the serum or fraction thereof relative to a cell that was not contacted with the serum or fraction thereof indicates that the serum or fraction thereof comprises an agent that increases the expression of a sirtuin gene. A transcriptional control region may be a promoter region. The sirtuin gene may encode SIRT1 or Sir2 from yeast. Also provided are compositions comprising a fraction from the serum of a non-calorically-restricted organism, wherein the fraction of serum inhibits the expression of a sirtuin. A composition may be enriched in an agent that suppresses the expression of a sirtuin, identified by a method comprising, e.g., (i) obtaining one or more fractions of serum from a non-calorically-restricted organism; (ii) contacting the one or more fractions with a cell; (iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin; and (iv) obtaining one or more subfractions of the first fraction that contains an agent that inhibits the expression of a sirtuin and repeating steps (ii) and (iii). The method may further comprise repeating step (iv).

Methods describe herein also include methods comprising contacting serum from a non-calorically-restricted organism with a cell and determining the level of expression of a sirtuin in the cell. A method may be used for determining the presence of an agent that inhibits the expression of a sirtuin in serum of a fraction thereof from a non-calorically-restricted organism, and may comprise (i) contacting a cell with a composition comprising serum or a fraction thereof from a non-calorically-restricted organism; and (ii) determining the level of expression of a sirtuin in the cell; wherein a lower level of sirtuin expression in the cell contacted with the composition comprising serum or a fraction thereof from a non-calorically-restricted organism relative to a composition comprising serum from a calorically-restricted organism or another fraction of the serum of the non-calorically-restricted organism indicates that the serum or fraction thereof from a non-calorically-restricted organism comprises an agent that induces the expression of a sirtuin. A method may also be used for enriching a composition in an agent that inhibits the expression of a sirtuin, and may comprise (i) obtaining one or more fractions of serum from a non-calorically-restricted organism; (ii) contacting the one or more fractions with a cell; (iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin; and (iv) obtaining one or more subfractions of the first fraction that contains an agent that inhibits the expression of a sirtuin and repeating steps (ii) and (iii). A method may further comprising repeating step (iv). A method may also be used for identifying a factor in serum from a non-calorically-restricted organism that inhibits the expression of a sirtuin, and may comprise (i) obtaining one or more fractions of serum from a non-calorically-restricted organism; (ii) contacting the one or more fractions with a cell; (iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin; (iv) obtaining one or more subfractions of the first fraction that contains an agent that inhibits the expression of a sirtuin and repeating steps (ii) and (iii); and (v) repeating steps (i)-(iv) for a number of times sufficient to identify the factor that inhibits the expression of a sirtuin.

A method for inhibiting the expression of a sirtuin in a cell and/or for increasing the susceptibility of a cell to apoptosis may comprise contacting the cell with serum or a fraction thereof from a non-calorically-restriction organism. The method may further comprise determining the level of expression of a sirtuin, e.g., SIRT1, or an ortholog thereof, in the cell. The fraction may be a composition enriched in an agent present in serum from a non-calorically-restricted organism. The method may be used for increasing the susceptibility of a cell to apoptosis and may further comprise decreasing the protein or activity level of Ku70 in the cell. Also provided are methods for identifying an agent that decreases the expression of a sirtuin gene. A method may comprise: (i) contacting a cell comprising a reporter gene operably linked to a transcriptional control region of a sirtuin gene with serum or a fraction thereof from a non-calorically-restricted organism; and (ii) determining the level of expression of the reporter gene, (iii) wherein a lower level of expression of the reporter gene in the cell contacted with the serum or fraction thereof relative to a cell that was not contacted with the serum or fraction thereof indicates that the serum or fraction thereof comprises an agent that decreases the expression of a sirtuin gene. The transcriptional control region may be a promoter region. The sirtuin gene may encode SIRT1 or Sir2.

Also provided are methods, e.g., diagnostic methods. These may be used for determining whether a subject is calorically-restricted or resistant to a stress condition. A method may comprise determining the protein level of a factor in the subject, wherein a higher level of the factor in the subject relative to that of a control subject indicates that the subject is calorically-restricted or resistant to stress. The method may comprise determining the level of a sirtuin protein, e.g., SIRT1, in a tissue of the subject. The method may also comprise determining the level of a factor in the serum of the subject, wherein the factor increases the level of expression of a sirtuin, e.g., SIRT1.

Also provided are compositions comprising serum from a calorically-restricted or non-calorically-restricted organism and a cell, e.g., a 293T or human embryonic kidney cell. Other compositions comprise serum from a calorically-restricted or non-calorically restricted organism and a cell. In certain embodiments, the organism is a mammal, e.g., a human. In certain embodiments, the organism is not a rat or a monkey.

### BRIEF DESCRIPTION OF THE DRAWINGS

FIG. 1. Caloric restriction increases SIRT1 expression in a variety of rat tissues and inhibits Bax-mediated apoptosis. (A) Twelve month old, male Fisher 344 rats were fed NIH-31 standard feed ad libitum (AL) or subjected to lifelong restriction starting immediately after weaning, with a daily food allotment of 60% of that eaten by the AL animals (CR). Water was available ad libitum for both groups. After sacrificing the animal, protein extracts from the liver, kidney, abdominal pads of adipose tissue, and the brain were prepared as described in (29). Extracts of tissues (˜25 μg) from three AL and three CR animals were separated by SDS-PAGE and probed with a rabbit polyclonal antibody against SIRT1, or monoclonal antibody against β-actin. (B) Total protein extracts (50 μg) from human embryonic kidney 293 cells were separated by SDS-PAGE and probed for SIRT1. β-actin served as a loading control. (C) 293T cells were grown in DME media containing 10% serum from either AL rats or CR rats as above. After 24 hrs, cells were transfected with YFP (1 μg), YFP-Bax (1 μg) or YFP-Bax (1 μg) and Ku70 (2 μg). The percentage of YFP positive cells with apoptotic nuclei was scored 24 hrs post-transfection. Values represent the average of three experiments in which at least 200 cells were counted. Error bars represent S.E.M.

FIG. 2. Attenuation of apoptosis by CR serum and the interaction between Ku70 and Bax is SIRT1-dependent. (A) 293 cells and 293 cells stably expressing the dominant negative SIRT1 H363Y mutation, were grown in DME media containing 10% serum from either AL or CR rats as described above. After 24 hrs, cells were transfected with YFP-Bax (1 μg) and Ku70 (2 μg). Percent apoptosis was determined 24 hrs post-transfection. (B) 293 cells were grown in AL or CR media and were transfected with either siRNA vector or siRNA-SIRT1 vector (1 μg). Twenty-four hrs post-transfection, the cells were again transfected with siRNA empty vector or siRNA-SIRT1 (1 μg) along with YFP, YFP-Bax or YFP-Bax and Ku70, as above. The percentage YFP-positive cells with apoptotic nuclei was scored 24 hrs following the second transfection. Statistical significance was determined by ANOVA. (C) Endogenous Ku70 and Bax from 293 cells or 293 cells stably expressing the H363Y SIRT1 allele were co-immunoprecipitated in Chaps buffer (150 mM sodium chloride, 10 mM Hepes pH 7.4, 1.0% CHAPS) and analyzed by Western blotting.

### DETAILED DESCRIPTION

### Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

“Activating a sirtuin protein” refers to the action of producing an activated sirtuin protein, i.e., a sirtuin protein that is capable of performing at least one of its biological activities to at least some extent, e.g., with an increase of activity of at least about 10%, 50%, 2 fold or more. Biological activities of sirtuin proteins include deacetylation, e.g., of histones and p53; extending lifespan; increasing genomic stability; silencing transcription; and controlling the segregation of oxidized proteins between mother and daughter cells.

An “agent” or “factor” refers to any molecule or complex of molecules. A molecule or complex of molecules can be, e.g., a protein, a peptide, a nucleic acid, or a small organic molecule. An agent can be a growth factor, such as insulin-like growth factor (IGF)-1 or insulin. An agent may also be nicotinamide phosphoribosyltransferase (NAMPRT; E.C.2.4.2.12).

“Bax” refers to Bcl-2 Associated X protein. Bax is a proapoptotic protein that induces cell death by acting on mitochondria. Six alternatively spliced transcript variants, which encode different isoforms, have been reported for this gene. Exemplary nucleotide and amino acid sequences of human Bax isoform a include NM_{—}138761 and NP_{—}620116, respectively. Exemplary nucleotide and amino acid sequences of human Bax isoform β include NM_{—}004324 and NP_{—}004315, respectively. Exemplary nucleotide and amino acid sequences of human Bax protein isoform γ include NM_{—}138762 and NP_{—}620117, respectively. Exemplary nucleotide and amino acid sequences of human Bax isoform δ include NM_{—}138763 and NP_{—}620118, respectively. Exemplary nucleotide and amino acid sequences of human Bax isoform ε include NM_{—}138764 and NP_{—}620119, respectively. Exemplary nucleotide and amino acid sequences of human Bax isoform σ include NM_{—}138765 and NP_{—}620120, respectively.

A “calorically-restricted organism” refers to an organism that is on a calorically-restricted or “CR” diet, i.e., a diet according to which an organism is fed less than its ad libitum diet, e.g., 50 to 70% of an ad libitum diet. For example, a calorically-restricted diet can consist of 60% of the food intake relative to an ad libitum diet. In particular species, a calorically-restricted diet can also consist of a reduction of one nutrient relative to others. For example, calorically-restricted yeast may consist of yeast that is on a diet in which glucose or non-essential amino acids are reduced 50-80%. In flies, a CR diet may consist of lowering amino acids in the food or reducing the yeast concentration in the agar by more than 2 fold (aka dietary restriction). In rats, a CR diet may consist of reducing caloric intake 15-40% from what a rodent eats ad libitum without any deficiencies in nutrients. In monkeys, a CR diet may consist in reducing caloric intake 15-40% from what a monkey eats ad libitum without any deficiencies in nutrients. In humans, a CR diet may consist of reducing caloric intake 20-30% from a standard healthy diet for that person's age and weight without any deficiencies in nutrients. A “non-calorically-restricted organism” refers to an organism that is fed ad libitum.

A “composition enriched in an agent” refers to a composition in which the agent has been concentrated at least about 2, 5, 10, 30, 100, 300, or a 1000 times.

The terms “comprise” and “comprising” are used in the inclusive, open sense, meaning that additional elements may be included.

“Determining the level of expression of a sirtuin” refers to determining the level of the sirtuin protein or mRNA encoding such.

A “fraction of serum” refers to a portion of the serum, such as a portion obtained following any fractionation method, e.g., size fractionation, or affinity purification. A “subfraction” refers to a fraction of a fraction. A fraction of serum can be about 50% of the serum; about 5%; 1%; 10⁻¹%; 10⁻²%; 10⁻³% or less of the serum, by volume or by weight.

The term “including” is used to mean “including but not limited to”. “Including” and “including but not limited to” are used interchangeably.

To “induce the expression of a sirtuin” refers to increasing the protein level of a sirtuin. This can occur, e.g., by increasing the transcription of a gene encoding the sirtuin, by stabilizing the mRNA encoding the sirtuin or by stabilizing the sirtuin protein. Induction can be by a factor of about 50%, two fold, three fold, five fold, 10 fold, 100 fold or more.

“Inhibiting a sirtuin protein” refers to the action of reducing at least one of the biological activities of a sirtuin protein to at least some extent, e.g., at least about 10%, 50%, 2 fold or more.

“Ku70” refers to a DNA end-joining protein that was first characterized as part of the Ku70/Ku80 heterodimer. Exemplary nucleotide and amino acid sequences of human Ku70 are set forth as SEQ ID NOs: 19 and 20, corresponding to GenBank™ Accession Numbers: NM_{—}001469 and NP_{—}001460, respectively. Genomic sequences can be found in GenBank Accession numbers NT_{—}011520 and AC144560.3. Exemplary nucleotide and amino acid sequences of mouse Ku70 are GenBank™ Accession Numbers: NM_{—}010247, NP_{—}034377, AH006747, and NT_{—}081922. Exemplary nucleotide and amino acid sequences of rat Ku70 are GenBank™ Accession Numbers: NM_{—}139080, NP_{—}620780, AB066102, and NW_{—}047781. The Ku70/Ku80 heterodimer is essential for the repair of DNA double strand breaks by nonhomologous end joining as well as the rearrangement of antibody and T cell receptor genes via V(D)J recombination (Featherstone et al., Mutat. Res. 434:3-15 (1999)).

“Nicotinamide phosphribosyltransferase” (NAMPRT; E.C.2.4.2.12) is also known as pre-B-cell colony enhancing factor 1 (PBEF1) and visfatin, and exists as two isoforms (see, e.g., Samal et al. (1994) Mol. Cell. Biol. 14:1431, Rongwaux et al. (2002) Euro. J. Immunol. 32:3225 and Fukuhara et al. Science 307:426-30 (2005); U.S. Pat. Nos. 5,874,399 and 6,844,163). The sequence of isoform a is available under Genbank Accession numbers NM_{—}005746, NP_{—}005737 and U02020 and the sequence of isoform b is available under GenBank Accession numbers NM_{—}182790, NP_{—}877591 and BC020691. The nucleotide and amino acid sequences of human NAMPRT isoform a (NM_{—}005746) are set forth as SEQ ID NOs: 21 and 22. The nucleotide and amino acid sequences of human NAMPRT isoform b (BC020691) are set forth as SEQ ID NOs: 23 and 23, respectively.

An “organism” or “subject” includes mammals as well as non-mammalian organisms. Mammals include humans and non-humans, e.g., ovines, bovines, sheep, horses, non-human primates, felines, canines, rats, and mice.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

To “reduce” or “inhibit the expression of a sirtuin” refers to reducing the protein level of a sirtuin by a factor of about 50%, two fold, three fold, five fold, 10 fold, 100 fold or more.

“Replicative lifespan” which is used interchangeably herein with “lifespan” of a cell refers to the number of daughter cells produced by an individual “mother cell.” “Chronological aging,” on the other hand, refers to the length of time a population of non-dividing cells remains viable when deprived of nutrients. “Increasing the lifespan of a cell” or “extending the lifespan of a cell,” as applied to cells or organisms, refers to increasing the number of daughter cells produced by one cell; increasing the ability of cells or organisms to cope with stresses and combat damage, e.g., to DNA, proteins; and/or increasing the ability of cells or organisms to survive and exist in a living state for longer under a particular condition, e.g., stress. Lifespan can be increased by at least about 20%, 30%, 40%, 50%, 60% or between 20% and 70%, 30% and 60%, 40% and 60% or more using methods described herein.

“Sirtuin deacetylase protein family members;” “Sir2 family members;” “Sir2 protein family members;” or “sirtuin proteins” includes yeast Sir2, Sir-2.1, and human SIRT1 and SIRT2 proteins. The nucleotide and amino acid sequences of the human sirtuin, SIRT1 (silent mating type information regulation 2 homolog), are set forth as SEQ ID NOs: 1 and 2, respectively (corresponding to GenBank Accession numbers NM_{—}012238 and NP_{—}036370, respectively). The mouse homolog of SIRT1 is Sirt20. Other family members include the four additional yeast Sir2-like genes termed “HST genes” (homologues of Sir two) HST1, HST2, HST3 and HST4, and the six other human homologues: hSIRT2 (corresponding to Genbank Accession numbers NM_{—}012237 and NP_{—}036369 for variant 1; SEQ ID NOs: 3 and 4, respectively; and NM_{—}030593 and NP_{—}085096 for variant 2; SEQ ID NOs: 5 and 6, respectively); hSIRT3 (corresponding to Genbank Accession numbers NM_{—}012239 and NP_{—}036371; SEQ ID NOs: 7 and 8, respectively); hSIRT4 (corresponding to Genbank Accession numbers NM_{—}012240 and NP_{—}036372; SEQ ID NOs: 9 and 10, respectively); hSIRT5 (corresponding to Genbank Accession numbers NM_{—}012241 and NP_{—}036373 for variant 1 (SEQ ID NOs: 11 and 12, respectively) and NM_{—}031244 and NP_{—}112534 for variant 2 (SEQ ID NOs: 13 and 14, respectively)); hSIRT6 (corresponding to Genbank Accession numbers NM_{—}016539 and NP_{—}057623; SEQ ID NOs: 15 and 16, respectively); and hSIRT7 (corresponding to Genbank Accession numbers NM_{—}016538 and NP_{—}057622; SEQ ID NOs: 17 and 18, respectively) (Brachmann et al. (1995) Genes Dev. 9:2888 and Frye et al. (1999) BBRC 260:273). Human sirtuins are represented by upper case letters with or without an “h” in front (i.e., HST or hHST). Preferred sirtuins are those that share more similarities with SIRT1, i.e., hSIRT1, and/or Sir2 than with hSIRT2, such as those members having at least part of the N-terminal sequence present in SIRT1 and absent in SIRT2 such as SIRT3 has. Yeast Sir2 amino acid sequence can be found at GenBank Accession No. P53685. C. elegans Sir-2.1 amino acid sequence can be found at Genbank Accession No. NP_{—}501912. Human SIRT1 genomic sequences can be found in any of the following GenBank entries (1) NT_{—}008583; (2) AADD01108281.1; (3) AADB01061226.1; (4) AADC01091523.1; and (5) AL133551.13. Mouse SIRT1 genomic sequences can be found at either of the following two GenBank entries (1) NT_{—}039495 and (2) CAAA01066320.1. Rat SIRT2 gene sequence (the homolog of human SIRT1) can be found, e.g., as GenBank Accession No. (1) NW_{—}047557. Human SIR12 genomic sequence can be found in any of the following GenBank entries: (1) NT_{—}0111009; (2) AADD01165337.1; (3) AADC01139123.1; and (4) AC011455.6.

“Stress” refers to any non-optimal condition for growth, development or reproduction. A “stress condition” can be exposure to heatshock; osmotic stress; a DNA damaging agent; inadequate salt level; inadequate nitrogen levels; inadequate nutrient level; radiation or a toxic compound, e.g., a toxin or chemical warfare agent (such as dirty bombs and other weapons that may be used in bioterrorism). “Inadequate levels” refer to levels that result in non-optimal condition for growth, development or reproduction.

“Substantially purified” refers to a protein that has been separated from components which naturally accompany it. Preferably the protein is at least about 80%, more preferably at least about 90%, and most preferably at least about 99% of the total material (by volume, by wet or dry weight, or by mole percent or mole fraction) in a sample. Purity can be measured by any appropriate method, e.g., in the case of polypeptides by column chromatography, gel electrophoresis or HPLC analysis.

A “transcriptional control region” of a gene refers to a portion of the gene that is involved in regulating, the level of transcription of the gene and can be, e.g., a promoter region (located 5′ of the transcription initiation site), an untranslated region, or at least a portion of an intron.

The term “treating” a condition or disease is art-recognized and refers to curing as well as ameliorating at least one symptom of a condition or disease or preventing the condition or disease from worsening.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

### Exemplary Compositions

Provided herein are compositions comprising serum or isolated serum, fractions or agents thereof, from a calorically-restricted or non-calorically-restricted organism. The organism can be a mammal, such as a human. In certain embodiments, the organism is a mammal with the proviso that the mammal is not a rat or a monkey. The organism can also be an insect, such as a fly or a worm. The cell can be a mammalian cell, such as a human cell, e.g., a 293T or a human embryonic kidney cell. The serum preferably contains an agent that modulates expression of a sirtuin, e.g., sir2 or SIRT1. Modulation of expression of a sirtuin can be shown by any of numerous methods known in the art.

Also provided herein are fractions of serum from a calorically-restricted or non-calorically-restricted organism. Fractions may consist of fractions that are enriched in an agent that modulates the expression of a sirtuin. A composition may comprise a fraction from the serum of a calorically-restricted organism, wherein the fraction of serum induces the expression of a sirtuin, e.g., relative to serum from a non-calorically-restricted organism or relative to another fraction of the serum of a calorically-restricted organism. A composition may also comprise a fraction from the serum of a non-calorically-restricted organism, wherein the fraction of serum inhibits the expression of a sirtuin, e.g., relative to serum from a calorically-restricted organism or relative to another fraction of serum from a non-calorically-restricted organism.

Other compositions provided herein are compositions that are enriched in an agent that induces the expression of a sirtuin, identified, e.g., by a method comprising one or more of the following steps: (i) obtaining one or more fractions of serum from a calorically-restricted organism; (ii) contacting the one or more fractions with a cell; (iii) determining the level of expression of a sirtuin in the cell, wherein a higher level of expression of the sirtuin in the cell contacted with a fraction relative to a cell that was not contacted with the fraction indicates that the fraction contains an agent that induces the expression of a sirtuin; and (iv) obtaining one or more subfractions of a fraction that contains an agent that induces the expression of a sirtuin and repeating steps (ii) and (iii). The method may further comprise repeating step (iv). Another composition may be enriched in an agent that suppresses the expression of a sirtuin, identified, e.g., by a method comprising one or more of the following steps: (i) obtaining one or more fractions of serum from a non-calorically-restricted organism; (ii) contacting the one or more fractions with a cell; (iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin; and (iv) obtaining one or more subfractions of the first fraction that contains an agent that inhibits the expression of a sirtuin and repeating steps (ii) and (iii). The method may further comprise repeating step (iv).

Other compositions comprise an isolated factor obtained from serum from a calorically-restricted or non-calorically-restricted organism that modulates the level of expression of a sirtuin. Yet other compositions comprise or consist of a combination of any of the compositions described herein. Compositions described herein may also be combined with other agents or compositions known to modulate (increase or decrease) the level or activity of sirtuins, such as those described in WO 05/002672.

Compositions also include those comprising serum, isolated serum or a fraction or agent thereof from a calorically-restricted or non-calorically-restricted organism and a cell. The cell can be a mammalian cell, such as a human cell, or a non-mammalian cell. In certain embodiments, the serum and cell do not normally occur together in nature. For example, the cell may be from an established cell line.

Also provided herein are methods for preparing a composition described herein, such as a method for isolating serum or a fraction thereof from a calorically-restricted or non-calorically-restricted organism. Other methods comprise contacting serum or a fraction thereof from a calorically-restricted organism and a cell.

### Exemplary Methods for Identifying or Purifying a Factor

Further provided are methods for determining the presence of an agent that induces the expression of a sirtuin in serum or a fraction thereof from a calorically-restricted organism, comprising (i) contacting a cell with a composition comprising serum or a fraction thereof from a calorically-restricted organism; and (ii) determining the level of expression of a sirtuin in the cell; wherein a higher level of sirtuin expression in the cell contacted with the composition comprising serum or a fraction thereof from a calorically-restricted organism relative to, e.g., a composition that does not comprise serum from a calorically-restricted organism or relative to another fraction of serum from the calorically-restricted organism, indicates that the serum or fraction thereof from the calorically-restricted organism comprises an agent that induces the expression of a sirtuin.

A method for determining the presence of an agent that inhibits the expression of a sirtuin in serum or a fraction thereof from a non-calorically-restricted organism may comprise (i) contacting a cell with a composition comprising serum or a fraction thereof from a non-calorically-restricted organism; and (ii) determining the level of expression of a sirtuin in the cell; wherein a lower level of sirtuin expression in the cell contacted with the composition comprising serum or a fraction thereof from a non-calorically-restricted organism relative to, e.g., a composition comprising serum from a calorically-restricted organism or another fraction of the serum of the non-calorically-restricted organism indicates that the serum or fraction thereof from a non-calorically-restricted organism comprises an agent that induces the expression of a sirtuin.

A method for enriching a composition in an agent that induces the expression of a sirtuin may comprise one or more of the following steps: (i) obtaining one or more fractions of serum from a calorically-restricted organism; (ii) contacting the one or more fractions with a cell; (iii) determining the level of expression of a sirtuin in the cell, wherein a higher level of expression of the sirtuin in the cell contacted with a first fraction relative to a cell that was not contacted with the fraction or contacted with a second fraction indicates that the first fraction contains an agent that induces the expression of a sirtuin; and (iv) obtaining one or more subfractions of the fraction that contains an agent that induces the expression of a sirtuin and repeating steps (ii) and (iii), to thereby enrich a composition in an agent that induces the expression of a sirtuin. The method may further comprise repeating step (iv).

A method for enriching a composition in an agent that inhibits the expression of a sirtuin may comprise one or more of the following steps: (i) obtaining one or more fractions of serum from a non-calorically-restricted organism; (ii) contacting the one or more fractions with a cell; (iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin; and (iv) obtaining one or more subfractions of the first fraction that contains an agent that inhibits the expression of a sirtuin and repeating steps (ii) and (iii). The method may further comprise repeating step (iv).

A method for identifying a factor in serum from a calorically-restricted organism that induces the expression of a sirtuin may comprise one or more of the following steps: (i) obtaining one or more fractions of serum from a calorically-restricted organism; (ii) contacting the one or more fractions with a cell; (iii) determining the level of expression of a sirtuin in the cell, wherein a higher level of expression of the sirtuin in the cell contacted with a first fraction relative to a cell that was not contacted with the fraction or contacted with a second fraction indicates that the first fraction contains an agent that induces the expression of a sirtuin; (iv) obtaining one or more subfractions of the fraction that contains an agent that induces the expression of a sirtuin and repeating steps (ii) and (iii); and (v) repeating steps (i)-(iv) for a number of times sufficient to allow identification the factor that induces the expression of a sirtuin. The number of times may be 2, 3, 5, 10, 20, 50 or more. A factor (or agent) may be identified by any of numerous well known methods, e.g., protein sequencing and mass spectrometry. Accordingly, a number of times sufficient to allow identification of the factor refers to a number of times sufficient to produce the factor (or agent) in sufficiently pure form for a particular technique of identification.

A method for identifying a factor in serum from a non-calorically-restricted organism that inhibits the expression of a sirtuin may comprise one or more of the following steps: (i) obtaining one or more fractions of serum from a non-calorically-restricted organism; (ii) contacting the one or more fractions with a cell; (iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin; (iv) obtaining one or more subfractions of the first fraction that contains an agent that inhibits the expression of a sirtuin and repeating steps (ii) and (iii); and (v) repeating steps (i)-(iv) for a number of times sufficient to identify the factor that inhibits the expression of a sirtuin.

Other methods or the methods described herein may also be modified or complemented by adding to the serum or fraction thereof of calorically-restricted or non-calorically-restricted organisms fractions from other types of serum or purified factors. Fractions of serum from a calorically-restricted animal can be combined with fractions of serum from a non-calorically-restricted animal and the effect on sirtuin expression in a cell determined. Serum from non-calorically-restricted organisms may also be combined with a growth factor, e.g., IGF-1 or insulin, and the level of expression of a sirtuin may be determined. Thus, methods for identifying the presence of an agent in serum from non-calorically-restricted organisms that inhibits expression of a sirtuin may comprise contacting the serum with known factors, such as growth or differentiation factors, and determining the effect on the expression of the sirtuin.

### Exemplary Methods of Treatment of Cells and Organisms

Other methods provided herein comprise methods for modulating, e.g., increasing or decreasing the expression of a sirtuin in a cell. An exemplary method comprises contacting the cell with serum or a fraction thereof from a calorically-restricted organism. Another exemplary method comprises contacting the cell with a composition enriched in an agent present in serum from a calorically-restricted organism. A method for inhibiting the expression of a sirtuin in a cell may comprise contacting the cell with serum or a fraction thereof from a non-calorically-restriction organism. Alternatively, the method comprises contacting the cell with a composition enriched in an agent present in serum from a non-calorically-restricted organism. These methods optionally include determining the level of expression of a sirtuin.

Also provided herein are methods for modulating the susceptibility of a cell to apoptosis, modulating lifespan extension, inhibiting tumor growth or size, modulating the resistance of a cell or organism to stress. For example, one method for reducing the susceptibility of a cell to apoptosis may comprise contacting the cell with serum or a fraction thereof from a calorically-restricted organism. A method for reducing the susceptibility of a cell to apoptosis may also comprise contacting the cell with a composition enriched in an agent present in serum from a calorically-restricted organism. Another method for reducing the susceptibility of a cell to apoptosis may comprise contacting the cell with serum or a fraction thereof from a calorically-restricted organism and increasing the protein or activity level of Ku70 protein in the cell.

Methods for increasing the susceptibility of a cell to apoptosis may comprise contacting the cell with serum from a non-calorically-restricted organism. Alternatively, a method for increasing the susceptibility of a cell to apoptosis may comprise contacting the cell with a composition enriched in an agent present in serum from a non-calorically-restricted organism. A method for increasing the susceptibility of a cell to apoptosis may also comprise contacting the cell with serum or a fraction thereof from a non-calorically-restricted organism and decreasing the protein or activity level of Ku70 protein in the cell.

The protein level of Ku70 can be modulated according to methods known in the art. It can be increased by, e.g., overexpressing a nucleic acid encoding Ku70. Exemplary methods are described, e.g., in Cohen et al. Science 305: 390-392 (2004) and Cohen et al. Mol. Cell. 13:627-38 (2004).

Once an agent whose presence in serum from calorically-restricted organisms or animals increases the level of sirtuins in cells or whose presence in serum from non-calorically-restricted organisms or animals decreases the level of sirtuins in cells has been identified, the expression of sirtuins in cells can be modulated by modulating the level or activity of such an agent. For example, such a factor may be contacted with a cell or administered to a subject in need thereof. Drugs that block the synthesis of such factors or that sequester them to particular cell or bodily locations may also be used and further developed.

Serum, fractions thereof, agents identified and/or purified as described herein, or drugs effecting the level or activity of these agents, can be used, e.g., for modulating the lifespan, susceptibility to apoptosis and stress resistance of cells and organisms. An agent or composition that increases the level of a sirtuin, e.g., SIRT1, may be used for mimicking calorie restriction and the effects thereof. For example, the agents can be used, e.g., in treating or preventing diseases, e.g., diseases of aging, such as cancer, diabetes, atherosclerosis, catexia, cataracts, autoimmune and neurological disorders. Agents can also be used as food supplements to render subjects to whom they are administered more resistant to stress and potentially to increase their lifespan. Agents could also be used in vitro or ex vivo, e.g., for providing cell or organ survival and stress resistance. Generally, an agent that stimulates sirtuin expression can be used to extend the lifespan of cells, to render cells more resistant to stress, and to reduce apoptosis. On the contrary, an agent that inhibits sirtuin expression can be used to reduce the lifespan of cells, to render cells more sensitive to stress and to stimulate apoptosis.

In one embodiment, cells are treated in vitro as described herein to mimic caloric restriction, such as to extend their lifespan, e.g., to keep them proliferating longer and/or increasing their resistance to stress or prevent apoptosis. This is particularly useful for primary cell cultures (i.e., cells obtained from an organism, e.g., a human), which are known to have only a limited lifespan in culture. Treating such cells according to methods described herein, e.g., by contacting them with a composition (consisting of or comprising, e.g., serum, a fraction thereof, a purified agent or a pure agent) that increases sirtuin expression, will result in increasing the amount of time that the cells are kept a live in culture. Embryonic stem (ES) cells and pluripotent cells, and cells differentiated therefrom, can also be treated according to the methods described herein such as to keep the cells or progeny thereof in culture for longer periods of time. Primary cultures of cells, ES cells, pluripotent cells and progeny thereof can be used, e.g., to identify compositions having particular biological effects on the cells or for testing the toxicity of compositions on the cells (i.e., cytotoxicity assays). Such cells can also be used for transplantation into a subject, e.g., after ex vivo modification.

In other embodiments, cells that are intended to be preserved for long periods of time are treated as described herein. The cells can be cells in suspension, e.g., blood cells, serum, biological growth media, or tissues or organs. For example, blood collected from an individual for administering to an individual can be treated as described herein, such as to preserve the blood cells for longer periods of time, such as for forensic purposes. Other cells that one may treat for extending their lifespan or protect against apoptosis include cells for consumption, e.g., cells from non-human mammals (such as meat), or plant cells (such as vegetables).

Generally, sirtuin-inducing compositions may be used for extending the lifespan of a cell; extending the proliferative capacity of a cell; slowing ageing of a cell; promoting the survival of a cell; delaying cellular senescence in a cell; or mimicking the effects of calorie restriction.

Compositions may also be applied during developmental and growth phases in mammals, plants, insects or microorganisms, in order to, e.g., alter, retard or accelerate the developmental and/or growth process.

In another embodiment, cells obtained from a subject, e.g., a human or other mammal, are treated according to methods described herein and then administered to the same or a different subject. Accordingly, cells or tissues obtained from a donor for use as a graft can be treated as described herein prior to administering to the recipient of the graft. For example, bone marrow cells can be obtained from a subject, treated ex vivo, e.g., to extend their lifespan, and then administered to a recipient. The graft can be an organ, a tissue or loose cells.

In yet other embodiments, cells are treated in vivo, e.g., to increase their lifespan or prevent apoptosis. For example, skin can be protected from aging, e.g., developing wrinkles, by treating skin, e.g., epithelial cells, as described herein. In an exemplary embodiment, skin is contacted with a pharmaceutical or cosmetic composition comprising a composition described herein. Exemplary skin afflictions or skin conditions include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiform and erythema nodosum), damage caused by the sun or other light sources, discoid lupus erythematosus, dermatomyositis, skin cancer and the effects of natural aging. The formulations may be administered topically, to the skin or mucosal tissue, as an ointment, lotion, cream, microemulsion, gel, solution or the like, as further described herein, within the context of a dosing regimen effective to bring about the desired result. A dose of active agent may be in the range of about 0.005 to about 1 micromoles per kg per day, preferably about 0.05 to about 0.75 micromoles per kg per day, more typically about 0.075 to about 0.5 micromoles per kg per day. It will be recognized by those skilled in the art that the optimal quantity and spacing of individual dosages will be determined by the nature and extent of the condition being treated, the site of administration, and the particular individual undergoing treatment, and that such optimums can be determined by conventional techniques. That is, an optimal dosing regimen for any particular patient, i.e., the number and frequency of doses, can be ascertained using conventional course of treatment determination tests. Generally, a dosing regimen involves administration of the topical formulation at least once daily, and preferably one to four times daily, until symptoms have subsided.

Topical formulations may also be used as preventive, e.g., chemopreventive, compositions. When used in a chemopreventive method, susceptible skin is treated prior to any visible condition in a particular individual.

Compositions can also be delivered locally, e.g., to a tissue or organ within a subject, such as by injection, e.g., to extend the lifespan of the cells; protect against apoptosis or induce apoptosis.

Generally, sirtuin-inducing compositions may be used in methods for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the disease or condition does not result from oxidative stress. In certain embodiments, a method does not significantly increase the resistance of the subject to oxidative stress. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a sirtuin-inducing composition is administered to a subject, such as to generally increase the lifespan of its cells and to protect its cells against stress and/or against apoptosis. It is believed that treating a subject with a composition described herein is similar to subjecting the subject to hormesis, i.e., mild stress that is beneficial to organisms and may extend their lifespan. For example, a composition can be taken by subjects as a food or dietary supplement. In one embodiment, such a composition is a component of a multi-vitamin complex. Compositions can also be added to existing formulations that are taken on a daily basis, e.g., statins and aspirin. Compositions may also be used as food additives.

Compositions described herein could also be taken as one component of a multi-drug complex or as a supplement in addition to a multi-drug regimen. In one embodiment, this multi-drug complex or regimen would include drugs or compositions for the treatment or prevention of aging-related diseases, e.g., stroke, heart disease, arthritis, high blood pressure, Alzheimer's. In another embodiment, this multi-drug regimen would include chemotherapeutic drugs for the treatment of cancer. In a specific embodiment, a composition could be used to protect non-cancerous cells from the effects of chemotherapy.

Sirtuin-inducing compositions may be administered to a subject to prevent aging and aging-related consequences or diseases, such as stroke, heart disease, such as heart failure, arthritis, high blood pressure, and Alzheimer's disease. Other conditions that can be treated include ocular disorders, e.g., associated with the aging of the eye, such as cataracts, glaucoma, and macular degeneration. Sirtuin-inducing compositions described herein can also be administered to subjects for treatment of diseases, e.g., chronic diseases, associated with cell death, such as to protect the cells from cell death. Exemplary diseases include those associated with neural cell death (e.g., neurodegenerative diseases) or muscular cell death, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections.

Cardiovascular diseases that can be treated or prevented include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to the retinal arterioles, the glomerular arterioles, the vasa nervorum, cardiac arterioles, and associated capillary beds of the eye, the kidney, the heart, and the central and peripheral nervous systems.

The compositions may also be used for increasing HDL levels in plasma of an individual.

Yet other disorders that may be treated with compositions that induce the expression of a sirtuin include restenosis, e.g., following coronary intervention, and disorders relating to an abnormal level of high density and low density cholesterol. Sirtuin inducer compositions may also be used for treating or preventing viral infections, such as infections by influenza, herpes or papilloma virus. They may also be used as antifungal agents, anti-inflammatory agents, neuroprotective agents, and agents for preventing or treating excessive weight, obesity, prediabetic conditions, insulin resistance, hyperinsulinemia, hyperproinsulinemia, delayed insulin release, dyslipidemia, hyperglycemia, impaired glucose tolerance, diabetes (e.g., diabetes type II), metabolic syndrome, syndrome X, retinopathy, peripheral neuropathy, nephropathy, hypertension, and other coronary artery diseases (CADs) and consequences thereof.

Sirtuin-inducing compositions described herein can also be administered to a subject suffering from an acute disease, e.g., damage to an organ or tissue, e.g., a subject suffering from stroke or myocardial infarction or a subject suffering from a spinal cord injury. Compositions can also be used to repair an alcoholic's liver.

Sirtuin-inducing compositions can also be administered to subjects who have recently received or are likely to receive a dose of radiation. In one embodiment, the dose of radiation is received as part of a work-related or medical procedure, e.g., working in a nuclear power plant, flying an airplane, an X-ray, CAT scan, or the administration of a radioactive dye for medical imaging; in such an embodiment, the composition is administered as a prophylactic measure. In another embodiment, the radiation exposure is received unintentionally, e.g., as a result of an industrial accident, terrorist act, or act of war involving radioactive material. In such a case, the composition is preferably administered as soon as possible after the exposure to inhibit apoptosis and the subsequent development of acute radiation syndrome.

Compositions that reduce or inhibit sirtuin expression may be administered to a subject in conditions in which apoptosis of certain cells is desired. Higher a mounts of compositions that stimulate sirtuin expression relative to those that inhibit apoptosis may also be used for inducing apoptosis (see, e.g., WO 05/002672). For example, cancer may be treated or prevented. Exemplary cancers are those of the brain and kidney; hormone-dependent cancers including breast, prostate, testicular, and ovarian cancers; lymphomas, and leukemias. In cancers associated with solid tumors, a activating composition may be administered directly into the tumor. Cancer of blood cells, e.g., leukemia can be treated by administering a activating composition into the blood stream or into the bone marrow. Benign cell growth can also be treated, e.g., warts. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-1 associated malignant and benign disorders can also be treated by administration of compositions. Alternatively, cells can be obtained from a subject, treated ex vivo to remove certain undesirable cells, e.g., cancer cells, and administered back to the same or a different subject.

Chemotherapeutic agents that may be coadministered with compositions described herein as having anti-cancer activity (e.g., compositions that induce apoptosis; compositions that reduce lifespan or compositions that render cells sensitive to stress) include: aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, ocreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disrupters such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxin, dactinomycin, daunorubicin, docetaxel, doxorubicin, epirubicin, hexamethylmelamineoxaliplatin, iphosphamide, melphalan, mechlorethamine, mitomycin, mitoxantrone, nitrosourea, paclitaxel, plicamycin, procarbazine, teniposide, triethiylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compositions (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; chromatin disrupters.

These chemotherapeutic agents may be used by themselves with a composition described herein as inducing cell death or reducing lifespan or increasing sensitivity to stress and/or in combination with other chemotherapeutics agents. Many combinatorial therapies have been developed, including but not limited to those listed in Table 1.

TABLE 1
Exemplary conventional combination cancer chemotherapy

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, |
| | Vincristine, Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic lymphocytic | Chlorambucil, Prednisone |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, |
| | Melphalan |
| MAC-III | Methotrexate, Leucovorin, Dactinomycin, |
| | Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, Procarbazine, |
| | Hydroxyurea, Cisplatin, Cytarabine, Dacarbazine |

In addition to conventional chemotherapeutics, the compositions described herein as capable of inducing cell death or reducing lifespan can also be used with antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

The methods may be advantageous over combination therapies known in the art because they may allow conventional chemotherapeutic agents to exert greater effect at lower dosage. In a preferred embodiment, the effective dose (ED₅₀) for a chemotherapeutic agent or combination of conventional chemotherapeutic agents when used in combination with a composition described herein is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a composition described herein can be at least 2 fold greater than the TI for conventional chemotherapeutic regimen alone, and even more preferably at 5 fold, 10 fold or even 25 fold greater.

Sirtuin inhibitory compositions may also be used to stimulate weight gain, e.g., in subjects with cachexia.

Other combination therapies include conjoint administration with nicotinamide, NAD⁺ or salts thereof, or other Vitamin B3 analogs. Carnitines, such as L-carnitine, may also be co-administered, particularly for treating cerebral stroke, loss of memory, pre-senile dementia, Alzheimer's disease or preventing or treating disorders elicited by the use of neurotoxic drugs. Cyclooxygenase inhibitors, e.g., a COX-2 inhibitor, may also be co-administered for treating certain conditions described herein, such as an inflammatory condition or a neurologic disease.

Compositions or coformulations comprising a sirtuin inducer or inhibitor composition and another agent, e.g., a chemotherapeutic agent, an antiviral agent, nicotinamide, NAD⁺ or salts thereof, Vitamin B3 analogs, retinoids, alpha-hydroxy acid, ascorbic acid, are also encompassed herein.

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells. For example, activating compositions may be administered to farm animals to improve their ability to withstand farming conditions longer.

In other embodiments, methods described herein are applied to yeast cells. Situations in which it may be desirable to extend the lifespan of yeast cells include any process in which yeast is used, e.g., the making of beer, yogurt, and bakery items, e.g., bread. Use of yeast having an extended lifespan can result in using less yeast or in having the yeast be active for longer periods of time. Yeast or other mammalian cells used for recombinantly producing proteins may also be treated as described herein.

Compositions may also be used to increase lifespan, stress resistance, and resistance to apoptosis in plants. In one embodiment, a composition is applied to plants, e.g., on a periodic basis, or to fungi. In another embodiment, plants are genetically modified to produce a composition. In another embodiment, plants and fruits are treated with a composition prior to picking and shipping to increase resistance to damage during shipping. Plant seeds may also be contacted with compositions described herein, e.g., to preserve them.

Compositions may also be used to increase lifespan, stress resistance and resistance to apoptosis in insects. In this embodiment, compositions would be applied to useful insects, e.g., bees and other insects that are involved in pollination of plants. In a specific embodiment, a composition would be applied to bees involved in the production of honey. Generally, the methods described herein may be applied to any organism, e.g., eukaryote, that may have commercial importance. For example, they can be applied to fish (aquaculture) and birds (e.g., chicken and fowl).

Higher doses of compositions may also be used as a pesticide by interfering with the regulation of silenced genes and the regulation of apoptosis during development. In this embodiment, a composition may be applied to plants using a method known in the art that ensures the composition is bio-available to insect larvae, and not to plants.

At least in view of the link between reproduction and longevity (Longo and Finch, Science, 2002), the compositions can be applied to affect the reproduction of organisms such as insects, animals and microorganisms.

### Pharmaceutical Compositions and Methods

A method may comprise administering to a subject, e.g., a subject in need thereof, a therapeutically effective amount of a composition, e.g., a pharmaceutical composition, comprising an agent that modulates the level of expression of a sirtuin.

Pharmaceutical compositions for use in accordance with the present methods may be formulated in conventional manner using one or more physiologically acceptable carriers or excipients. Thus, activating compositions and their physiologically acceptable salts and solvates may be formulated for administration by, for example, injection, inhalation or insufflation (either through the mouth or the nose) or oral, buccal, parenteral or rectal administration. In one embodiment, the composition is administered locally, at the site where the target cells, e.g., diseased cells, are present, i.e., in the blood or in a joint.

Compositions can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compositions can be formulated in liquid solutions, preferably in physiologically compatible buffers such as Hank's solution or Ringer's solution. In addition, the compositions may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active composition.

For administration by inhalation, the compositions may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount Capsules and cartridges of e.g., gelatin, for use in an inhaler or insufflator may be formulated containing a powder mix of the composition and a suitable powder base such as lactose or starch.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions (including cosmetic preparations) may comprise from about 0.00001 to 100% such as from 0.001 to 10% or from 0.1% to 5% by weight of one or more compositions described herein.

In one embodiment, a composition described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Compositions may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, cited in the preceding section, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearic sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Compositions may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compositions useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor® Beiersdorf, Inc. (Norwalk, Conn.).

Compositions may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Compositions may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel, Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprylic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono-di- and triglycerides, mono- and di-esters of PEG (e.g., oleoyl macrogol glycerides), etc.

Compositions may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub. 10 MSO) and tetradecylmethyl sulfoxide; pyrrolidones such as 2-pyrrolidone, N-methyl-2-pyrrolidone and N—(-hydroxyethyl)pyrrolidone; urea; N,N-diethyl-m-toluamide; C.sub.2-C.sub.6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone® from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol®) and diethylene glycol monoethyl ether oleate (available commercially as Softcutol®); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol®); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., other anti-inflammatory agents, analgesics, antimicrobial agents, antifungal agents, antibiotics, vitamins, antioxidants, and sunblock a gents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprises an activating composition-containing microemulsion as described above, but may contain alternative pharmaceutically acceptable carriers, vehicles, additives, etc. particularly suited to oral or parenteral drug administration. Alternatively, an activating composition-containing microemulsion may be administered orally or parenterally substantially as described above, without modification.

Conditions of the eye can be treated or prevented by, e.g., systemic, topical, intraocular injection of a composition described herein, or by insertion of a sustained release device that releases a composition described herein.

Cells, e.g., treated ex vivo with a composition described herein, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Toxicity and therapeutic efficacy of compositions can be determined by standard pharmaceutical procedures in cell cultures or experimental animals. The LD₅₀ is the dose lethal to 50% of the population). The ED₅₀ is the dose therapeutically effective in 50% of the population. The dose ratio between toxic and therapeutic effects (LD₅₀/ED₅₀) is the therapeutic index. Compositions that exhibit large therapeutic indexes are preferred. While compositions that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage for use in humans. The dosage of such compositions may lie within a range of circulating concentrations that include the ED₅₀ with little or no toxicity. The dosage may vary within this range depending upon the dosage form employed and the route of administration utilized. For any composition, the therapeutically effective dose can be estimated initially from cell culture assays. A dose may be formulated in animal models to achieve a circulating plasma concentration range that includes the IC₅₀ (i.e., the concentration of the test composition that achieves a half-maximal inhibition of symptoms) as determined in cell culture. Such information can be used to more accurately determine useful doses in humans. Levels in plasma may be measured, for example, by high performance liquid chromatography.

### Exemplary Diagnostic Methods

Diagnostic methods are also provided herein. For example, one method may comprise determining whether a subject is calorically-restricted. The method may comprise determining the protein level of a sirtuin in the subject, e.g., in a cell of the subject, wherein a higher level of the sirtuin in the subject relative to that of a control subject indicates that the subject is calorically-restricted. The method may also comprise determining the level of a factor that modulates the level of expression of a sirtuin, e.g., a factor present in the serum of the subject, which factor increases the level of expression of a sirtuin in a cell. A control subject may be a healthy individual that is on an ad libitum diet. A higher level may be a level of at least about 50%, 2, 3, 5, 10, 30, or 100 fold.

A diagnostic method for determining whether a subject is calorically restricted may be combined with a method for treating a subject to reduce aging or aging-related diseases or conditions, e.g., as further described herein. For example, a method may comprise changing the diet of a subject, e.g., by reducing caloric intake, and determining the level of SIRT1 in a cell of the subject, wherein a higher level of SIRT1 in a cell of the subject after the change in diet relative to its level before the change of diet indicates that the subject is being treated to reduce aging or aging-related diseases or conditions.

Another diagnostic method may comprise determining whether a subject is more or less resistant to stress conditions relative to a control subject. An exemplary method comprises determining the protein level of a sirtuin in the subject, e.g., in a cell of the subject, wherein a higher level of the sirtuin in the subject relative to that of a control subject or value indicates that the subject is more resistant to stress conditions, whereas a lower level of the sirtuin in the subject relative to that of a control subject indicates that the subject is less resistant to stress conditions. The method may also comprise determining the level of a factor that modulates the level of expression of a sirtuin, e.g., a factor present in the serum of the subject, which factor increases the level of expression of a sirtuin in a cell. A higher or lower level may be a level of at least about 50%, 2, 3, 5, 10, 30, or 100 fold relative to that in the control subject.

A method that comprises measuring the level of a sirtuin in a subject may also be used to determine the general health of a subject and to monitor the general health of a subject who is receiving a treatment.

The level of a sirtuin may be measured in the blood or serum of the subject or in a tissue of the subject. A tissue may be fat tissue, such as comprising adipose cells and visceral fat, kidney tissue, liver tissue and brain tissue. The level of a sirtuin may also be determined in blood cells, skin cells, and hair cells (hair follicle).

The level of expression of a sirtuin may be determined by measuring the level of a sirtuin protein or transcript, such as mRNA. Protein levels may be determined according to conventional methods, e.g., using an antibody that binds specifically to a sirtuin.

### Exemplary Screening Methods

Other methods provided herein are methods for identifying an agent that modulates the expression of a sirtuin gene. In one embodiment, the method comprises (i) contacting a cell comprising a reporter gene operably linked to a transcriptional control region of a sirtuin gene with a test agent; and (ii) determining the level of expression of the reporter gene, wherein a different level of expression of the reporter gene in the cell contacted with the test agent relative to a cell that was not contacted with the test agent indicates that the test agent is an agent that modulates the expression of a sirtuin gene, e.g., a gene encoding SIRT1 or Sir2. The transcriptional control region may be a promoter region, such as having a nucleotide sequence provided in any of the GenBank Accession Nos set forth herein. The promoter region may comprise about 50, 100, 300, 500 or more nucleotides. An assay may further comprise determining whether a cell is calorically restricted or possesses one or more of the characteristics thereof, such as an extended lifespan or an increased resistance to stress. This may comprise measuring the level of a sirtuin, e.g., SIRT1 or Sirt2, in the cell comprising the reporter gene and contacted with the test agent. The assay may further comprise comparing the level of the sirtuin in the cell contacted with the test agent with the level of the sirtuin in a cell that was not contacted with the test agent.

A reporter gene assay as described herein may also comprise contacting a cell or cell lysate comprising a sirtuin regulatory sequence-reporter gene with serum or a fraction or purified factor thereof, from a calorically-restricted or non-calorically-restricted subject.

An assay for identifying an agent that modulates the expression of a sirtuin may comprise contacting a cell with a test agent and determining the level of expression of a sirtuin, such as with an antibody or a nucleic acid probe.

### Exemplary Kits

Also provided herein are kits, e.g., kits for therapeutic purposes or kits for modulating the lifespan of cells or modulating apoptosis. A kit may comprise one or more sirtuin-inducing or inhibitory compositions described herein, e.g., in premeasured doses. A kit may optionally comprise devices for contacting cells with the compositions and instructions for use. Devices include syringes, stents and other devices for introducing a composition into a subject or applying it to the skin of a subject.

Other kits may be used for diagnostic purposes. Exemplary kits may comprise an agent for determining the level of a sirtuin in a cell or tissue or the level of a serum factor that modulates the expression of a sirtuin. For example, a kit may comprise an antibody binding specifically to a sirtuin or to a factor inducing a sirtuin. Kits may also comprise probes, such as nucleic acid probes, for detecting the level of expression of a sirtuin. A kit may further comprise a device or means for obtaining a biological sample; a reagent for treating a biological sample for measuring the level of a sirtuin; or a secondary reagent, such as a secondary antibody. Controls, such as positive or negative controls may also be included, as well as a reference chart indicating levels of sirtuins in cells in particular conditions, e.g., a level of a sirtuin in a cell of a calorically restricted animal.

Another kit may comprise one or more reagents necessary for identifying and/or purifying and/or enriching a composition in a serum factor inducing the expression of a sirtuin.

The present description is further illustrated by the following examples, which should not be construed as limiting in any way. The contents of all cited references (including literature references, issued patents, published patent applications and GenBank Accession numbers, as cited throughout this application) are hereby expressly incorporated by reference.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Calorie Restriction Promotes Cell Survival by Inducing SIRT1

Given the role of yeast Sir2 in lifespan extension by CR (2, 3), we investigated whether the mammalian SIRT1, might also be involved in the CR response. Tissues were extracted from 12 month-old rats that had either been fed ad libitum (AL) or had been fed a CR diet (60% of AL) since weaning. 29. Animals were 12 month old, male Fisher 344 rats obtained from the animal vivarium at the Gerontology research center. Animals were fed NIH-31 standard feed. The CR animals were subjected to lifelong restriction, starting immediately after weaning, with a daily food allotment of 60% of that eaten by the AL animals. Water was available ad libitum for both groups. The animals were kept in a 12/12 hour light-dark cycle. Animals were sacrificed by decapitation early in the morning. Liver, kidney, abdominal pads of a dispose tissue, and the brain were removed and immediately frozen in liquid nitrogen. All procedures were approved by the Gerontology Research Center Animal Care and Use Committee: the NIA is AAALAC accredited. For analysis of SIRT1 protein levels, approximately 250 mg (brain, kidney, liver, muscle) or 1 g (adipose tissue) of tissue was homogenized in buffer C (2% lithium lauryl sulfate; 200 mM Tris-Cl, pH 7.5; 1 mM EDTA; phosphatase inhibitor cocktails; and a protease inhibitor cocktail). Following centrifugation the pellet and lipid layer (adipose tissue only) were discarded. An aliquot of the supernatant was set aside for use in protein assays, and the remaining samples were stored at −80° C. An aliquot of the supernatant was set aside for use in protein assays, and the remaining samples were stored at −80° C. For adipose tissue, an initial centrifugation was performed to allow removal of the lipid. Nonidet P40 was then added to a final concentration of 1%, as well as sodium deoxycholate to a final concentration of 0.25%. Following centrifugation (RCF=15,400) pellets were discarded and an aliquot of the supernatant was set aside for use in protein assays. Samples were stored at −80° C.

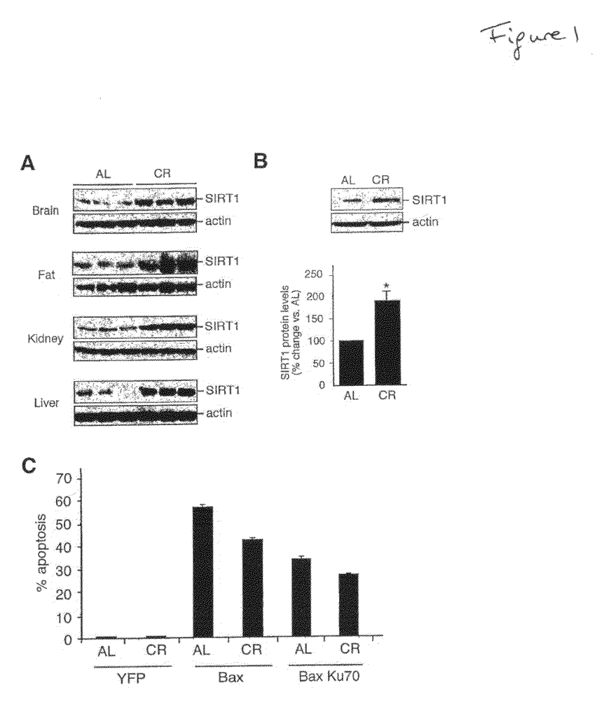

The results indicated that, compared to AL animals, the CR animals had significantly higher levels of SIRT1 protein in most tissues including brain, visceral fat pads, kidney, and liver (FIG. 1A).

To investigate whether SIRT1 might be responsible for the ability of CR to protect cells from stress-induced apoptosis, we utilized an in vitro cell culture model that recapitulates key in vivo proliferative and phenotypic features of CR (25). In this system, cells are cultured in the presence of serum from calorically-restricted rats, resulting in the induction of characteristic stress-response genes and the attenuation of stress-induced apoptosis (25). Consistent with our finding in tissues from calorically-restricted animals, SIRT1 levels were ˜2-fold higher in human embryonic kidney cells (293T) grown in the presence of CR versus AL serum (FIG. 1B).

To test the effect of CR serum on the susceptibility of cells to stress-induced apoptosis, we utilized a well-characterized assay whereby 293T cells are transfected with a Bax-YFP expression construct and YFP-positive cells are scored 24 hr later for a fragmented nucleus, a common marker of apoptosis (23, 24). 293T cells grown in serum from CR animals were less susceptible to Bax-transfection, compared to cells grown in media with AL serum, and this effect was enhanced by co-expressing Ku70 (FIG. 1C).

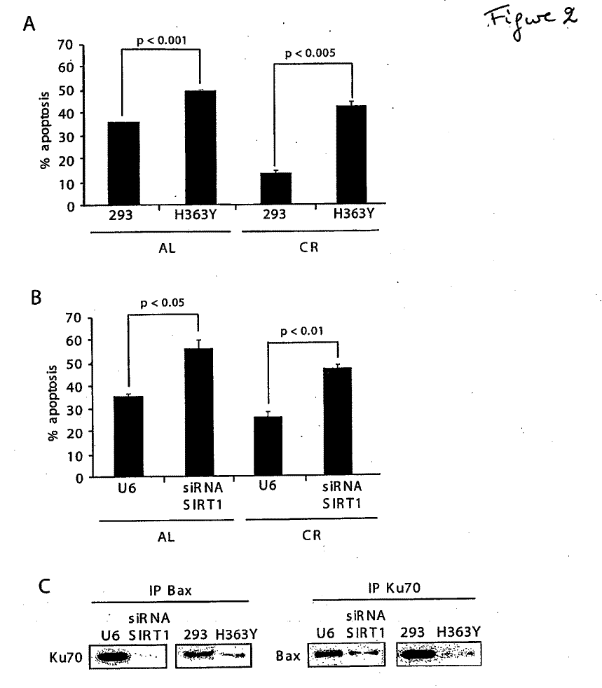

If CR serum attenuates apoptosis by boosting SIRT1 expression, then interfering with SIRT1 function should block its effect. To test this, we overexpressed the dominant negative SIRT1 allele SIRT1-H363Y, or siRNA against SIRT1 (pU6-siRNA-SIRT1 vector), and performed apoptosis assays as described above in the presence of AL or CR serum. Interference of SIRT1 function by either method severely abrogated the ability of CR to attenuate Bax-mediated apoptosis (FIGS. 2A, B). Both the dominant-negative and SIRT1 siRNA significantly abrogated the association between Ku70 and Bax (FIG. 2C).

Thus, CR induces SIRT1 expression in a wide array of tissues and this shifts the balance away from cell death towards cell survival. We postulate that in mammals, the induction of SIRT1 by CR represents a survival response that slows the age-dependent deterioration of physiological function by promoting the long-term survival of irreplaceable cells. It is interesting to note that the mammalian response to CR differs from that of budding yeast, in which Sir2 protein levels remain unchanged and enzymatic activity is regulated by small metabolites (3, 4). Calorically-restricted rodents and long-lived mutant mice are protected from cancer despite attenuating apoptosis (1, 22) possibly because (i) their cells possess heightened defenses and repair mechanisms (10, 14, 27) and (ii) they still retain the ability to undergo apoptosis if the damage is beyond repair (10). The induction of SIRT1 in visceral fat pads is particularly interesting in light of the recent demonstration that this tissue is involved in the regulation of rodent lifespan (28).

### REFERENCES

1. E. J. Masoro, Caloric restriction and aging: an update. Exp Gerontol 35, 299-305. (2000).
2. S. J. Lin, P. A. Defossez, L. Guarente, Requirement of NAD and SIR2 for life-span extension by calorie restriction in Saccharomyces cerevisiae. Science 289, 2126-8. (2000).
3. R. M. Anderson, K. J. Bitterman, J. G. Wood, O. Medvedik, D. A. Sinclair, Nicotinamide and Pnc1 govern lifespan extension by calorie restriction in S. cerevisiae. Nature 423, 181-5 (2003).
4. S. J. Lin, E. Ford, M. Haigis, G. Liszt, L. Guarente, Calorie restriction extends yeast life span by lowering the level of NADH. Genes Dev 18, 12-6 (2004).
5. M. Kaeberlein, A. A. Andalis, G. R. Fink, L. Guarente, High osmolarity extends life span in Saccharomyces cerevisiae by a mechanism related to calorie restriction. Mol Cell Biol 22, 8056-66 (2002).
6. S. Hekimi, L. Guarente, Genetics and the specificity of the aging process. Science 299, 1351-4 (2003).
7. M. Kaeberlein, M. McVey, L. Guarente, The SER2/3/4 complex and SIR2 alone promote longevity in Saccharomyces cerevisiae by two different mechanisms. Genes Dev 13, 2570-80. (1999).
8. H. A. Tissenbaum, L. Guarente, Increased dosage of a sir-2 gene extends lifespan in Caenorhabditis elegans. Nature 410, 227-30. (2001).
9. K. T. Howitz, K. J. Bitterman, H. Y. Cohen, D. W. Lamming, S. Lavu, J. G. Wood, R. E. Zipkin, P. Chung, A. Kisielewski, L. L. Zhang, B. Scherer, D. A. Sinclair, Small molecule activators of sirtuins extend Saccharomyces cerevisiae lifespan. Nature 425, 191-6 (2003).
10. A. Brunet, L. B. Sweeney, J. F. Sturgill, K. F. Chua, P. L. Greer, Y. Lin, H. Tran, S. E. Ross, R. Mostoslavsky, H. Y. Cohen, L. S. Hu, H. L. Cheng, M. P. Jedrychowski, S. P. Gygi, D. A. Sinclair, F. W. Alt, M. E. Greenberg, Stress-Dependent Regulation of FOXO Transcription Factors by the SIRT1 Deacetylase. Science (2004).
11. H. Vaziri, S. K. Dessain, E. N. Eaton, S. I. Imai, R. A. Frye, T. K. Pandita, L. Guarente, R. A. Weinberg, hSIR2(SIRT1) Functions as an NAD-Dependent p53 Deacetylase. Cell 107, 149-59. (2001).
12. J. Luo, A. Y. Nikolaev, S. Imai, D. Chen, F. Su, A. Shiloh, L. Guarente, W. Gu, Negative Control of p53 by Sir2alpha Promotes Cell Survival under Stress. Cell 107, 137-48. (2001).
13. E. P. M. Langley, Faretta M, Bauer U M, Frye R A, Minucci S, Pelicci P G, Kouzarides T., Human SIR2 deacetylates p53 and antagonizes PML/p53-induced cellular senescence. Embo J 21, 2383-2396 (2002).
14. M. C. Motta, N. Divecha, M. Lemieux, C. Kamel, D. Chen, W. Gu, Y. Bultsma, M. McBurney, L. Guarente, Mammalian SIRT1 represses forkhead transcription factors. Cell 116, 551-63 (2004).
15. K. Ando, Y. Higami, T. Tsuchiya, T. Kanematsu, I. Shimokawa, Impact of aging and life-long calorie restriction on expression of apoptosis-related genes in male F344 rat liver. Microsc Res Tech 59, 293-300 (2002).
16. Y. Higami, I. Shimokawa, Apoptosis in the aging process. Cell Tissue Res 301, 125-32 (2000).
17. H. R. Warner, Apoptosis: a two-edged sword in aging. Ann N Y Acad Sci 887, 1-11 (1999).
18. Y. Zhang, B. Herman, Ageing and apoptosis. Mech Ageing Dev 123, 245-60 (2002).
19. J. Campisi, Cellular senescence and apoptosis: how cellular responses might influence aging phenotypes. Exp Gerontol 38, 5-11 (2003).
20. D. C. Wallace, Mitochondrial diseases in man and mouse. Science 283, 1482-8 (1999).
21. R. R. Shelke, C. Leeuwenburgh, Lifelong caloric restriction increases expression of apoptosis repressor with a caspase recruitment domain (ARC) in the brain. Faseb J 17, 494-6 (2003).
22. E. Migliaccio, M. Giorgio, S. Mele, G. Pelicci, P. Reboldi, P. P. Pandolfi, L. Lanfrancone, P. G. Pelicci, The p66shc adaptor protein controls oxidative stress response and life span in mammals. Nature 402, 309-13. (1999).
23. M. Sawada, W. Sun, P. Hayes, K. Leskov, D. A. Boothman, S. Matsuyama, Ku70 suppresses the apoptotic translocation of Bax to mitochondria. Nat Cell Biol 5, 320-9 (2003).
24. H. Y. Cohen, S. Lavu, K. J. Bitterman, B. Hekking, T. A. Imahiyeiobo, C. Miller, R. Frye, H. Ploegh, B. M. Kessler, D. A. Sinclair, Acetylation of the C Terminus of Ku70 by CBP and PCAF Controls Bax-Mediated Apoptosis. Mol Cell 13, 627-38 (2004).
25. R. de Cabo, S. Furer-Galban, R. M. Anson, C. Gilman, M. Gorospe, M. A. Lane, An in vitro model of caloric restriction. Exp Gerontol 38, 631-9 (2003).
26. H. L. Cheng, R. Mostoslavsky, S. Saito, J. P. Manis, Y. Gu, P. Patel, R. Bronson, E. Appella, F. W. Alt, K. F. Chua, Developmental defects and p53 hyperacetylation in Sir2 homolog (SIRT1)-deficient mice. Proc Natl Acad Sci USA 100, 10794-9 (2003).
27. H. Tran, A. Brunet, J. M. Grenier, S. R. Datta, A. J. Fornace, Jr., P. S. DiStefano, L. W. Chiang, M. E. Greenberg, DNA repair pathway stimulated by the forkhead transcription factor FOXO3a through the Gadd45 protein. Science 296, 530-4 (2002).
28. M. Bluher, B. B. Kahn, C. R. Kahn, Extended longevity in mice lacking the insulin receptor in adipose tissue. Science 299, 572-4 (2003).

### EQUIVALENTS

Those skilled in the art will recognize, or be able to ascertain using no more than routine experimentation, many equivalents of the specific embodiments described herein. Such equivalents are intended to be encompassed by the following claims.

1. A composition comprising a fraction of serum of a calorically-restricted organism, wherein the fraction of serum induces the expression of a sirtuin.

2. The composition of claim 1 enriched in an agent that induces the expression of a sirtuin, identified by a method comprising
(i) obtaining one or more fractions of serum from a calorically-restricted organism;
(ii) contacting the one or more fractions with a cell;
(iii) determining the level of expression of a sirtuin in the cell, wherein a higher level of expression of the sirtuin in the cell contacted with a fraction relative to a cell that was not contacted with the fraction indicates that the fraction contains an agent that induces the expression of a sirtuin; and
(iv) obtaining one or more subfractions of a fraction that contains an agent that induces the expression of a sirtuin and repeating steps (ii) and (iii).

3. The composition of claim 2, wherein the method further comprises repeating step (iv).

4. A method comprising contacting serum from a calorically-restricted organism with a cell and determining the level of expression of a sirtuin in the cell.

5. The method of claim 4 for determining the presence of an agent that induces the expression of a sirtuin in serum or a fraction thereof from a calorically-restricted organism, comprising
(i) contacting a cell with a composition comprising serum or a fraction thereof from a calorically-restricted organism; and
(ii) determining the level of expression of a sirtuin in the cell;
wherein a higher level of sirtuin expression in the cell contacted with the composition comprising serum or a fraction thereof from a calorically-restricted organism relative to a composition that does not comprise serum from a calorically-restricted organism or relative to another fraction of serum from the calorically-restricted organism indicates that the serum or fraction thereof from the calorically-restricted organism comprises an agent that induces the expression of a sirtuin.

6. The method of claim 4 for enriching a composition in an agent that induces the expression of a sirtuin, comprising
i) obtaining one or more fractions of serum from a calorically-restricted organism;
(ii) contacting the one or more fractions with a cell;
(iii) determining the level of expression of a sirtuin in the cell, wherein a higher level of expression of the sirtuin in the cell contacted with a first fraction relative to a cell that was not contacted with the fraction or contacted with a second fraction indicates that the first fraction contains an agent that induces the expression of a sirtuin; and
(iv) obtaining one or more subfractions of the fraction that contains an agent that induces the expression of a sirtuin and repeating steps (ii) and (iii),
to thereby enrich a composition in an agent that induces the expression of a sirtuin.

7. The method of claim 6, further comprising repeating step (iv).

8. The method of claim 4 for identifying a factor in serum from a calorically-restricted organism that induces the expression of a sirtuin, comprising
i) obtaining one or more fractions of serum from a calorically-restricted organism;
(ii) contacting the one or more fractions with a cell;
(iii) determining the level of expression of a sirtuin in the cell, wherein a higher level of expression of the sirtuin in the cell contacted with a first fraction relative to a cell that was not contacted with the fraction or contacted with a second fraction indicates that the first fraction contains an agent that induces the expression of a sirtuin;
(iv) obtaining one or more subfractions of the fraction that contains an agent that induces the expression of a sirtuin and repeating steps (ii) and (iii); and
(v) repeating steps (i)-(iv) for a number of times sufficient to identify the factor that induces the expression of a sirtuin.

9. A method for increasing the expression of a sirtuin in a cell and/or reducing the susceptibility of a cell to apoptosis, comprising contacting the cell with serum or a fraction thereof from a calorically-restricted organism.

10. The method of claim 9, further comprising determining the level of expression of a sirtuin in the cell.

11. The method of claim 9, wherein the fraction is a composition enriched in an agent present in serum from a calorically-restricted organism.

12. The method of claim 9 for reducing the susceptibility of a cell to apoptosis, further comprising increasing the protein or activity level of Ku70 in the cell.

13. The method of claim 4 for identifying an agent that increases the expression of a sirtuin gene, comprising
(i) contacting a cell comprising a reporter gene operably linked to a transcriptional control region of a sirtuin gene with serum or a fraction thereof from a calorically-restricted organism; and
(ii) determining the level of expression of the reporter gene,
(iii) wherein a higher level of expression of the reporter gene in the cell contacted with the serum or fraction thereof relative to a cell that was not contacted with the serum or fraction thereof indicates that the serum or fraction thereof comprises an agent that increases the expression of a sirtuin gene.

14. The method of claim 13, wherein the transcriptional control region is a promoter region.

15. The method of claim 13, wherein the sirtuin gene encodes SIRT1.

16. The method of claim 13, wherein the sirtuin gene encodes sir2.

17. A composition comprising a fraction from the serum of a non-calorically-restricted organism, wherein the fraction of serum inhibits the expression of a sirtuin.

18. A composition of claim 17 enriched in an agent that suppresses the expression of a sirtuin, identified by a method comprising
(i) obtaining one or more fractions of serum from a non-calorically-restricted organism;
(ii) contacting the one or more fractions with a cell;
(iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin; and
(iv) obtaining one or more subfractions of the first fraction that contains an agent that inhibits the expression of a sirtuin and repeating steps (ii) and (iii).

19. The composition of claim 18, wherein the method further comprises repeating step (iv).

20. A method comprising contacting serum from a non-calorically-restricted organism with a cell and determining the level of expression of a sirtuin in the cell.

21. The method of claim 20 for determining the presence of an agent that inhibits the expression of a sirtuin in serum of a fraction thereof from a non-calorically-restricted organism, comprising
(i) contacting a cell with a composition comprising serum or a fraction thereof from a non-calorically-restricted organism; and
(ii) determining the level of expression of a sirtuin in the cell;
wherein a lower level of sirtuin expression in the cell contacted with the composition comprising serum or a fraction thereof from a non-calorically-restricted organism relative to a composition comprising serum from a calorically-restricted organism or another fraction of the serum of the non-calorically-restricted organism indicates that the serum or fraction thereof from a non-calorically-restricted organism comprises an agent that induces the expression of a sirtuin.

22. The method of claim 20 for enriching a composition in an agent that inhibits the expression of a sirtuin, comprising
(i) obtaining one or more fractions of serum from a non-calorically-restricted organism;
(ii) contacting the one or more fractions with a cell;
(iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin; and
(iv) obtaining one or more subfractions of the first fraction that contains an agent that inhibits the expression of a sirtuin and repeating steps (ii) and (iii).

23. The method of claim 22, further comprising repeating step (iv).

24. The method of claim 20 for identifying a factor in serum from a non-calorically-restricted organism that inhibits the expression of a sirtuin, comprising
(i) obtaining one or more fractions of serum from a non-calorically-restricted organism;
(ii) contacting the one or more fractions with a cell;
(iii) determining the level of expression of a sirtuin in the cell, wherein a lower level of expression of a sirtuin in the cell contacted with a first fraction relative to a cell that was contacted with serum from a calorically-restricted organism or relative to a cell that was contacted with a second fraction of serum from a non-calorically-restricted organism indicates that the first fraction contains an agent that inhibits the expression of a sirtuin;
(iv) obtaining one or more subfractions of the first fraction that contains an agent that inhibits the expression of a sirtuin and repeating steps (ii) and (iii); and
(v) repeating steps (i)-(iv) for a number of times sufficient to identify the factor that inhibits the expression of a sirtuin.

25. A method for inhibiting the expression of a sirtuin in a cell and/or for increasing the susceptibility of a cell to apoptosis, comprising contacting the cell with serum or a fraction thereof from a non-calorically-restriction organism.

26. The method of claim 25, further comprising determining the level of expression of a sirtuin in the cell.

27. The method of claim 25, wherein the fraction is a composition enriched in an agent present in serum from a non-calorically-restricted organism.

28. The method of claim 25 for increasing the susceptibility of a cell to apoptosis, further comprising decreasing the protein or activity level of Ku70 in the cell.

29. The method of claim 20 for identifying an agent that decreases the expression of a sirtuin gene, comprising
(i) contacting a cell comprising a reporter gene operably linked to a transcriptional control region of a sirtuin gene with serum or a fraction thereof from a non-calorically-restricted organism; and
(ii) determining the level of expression of the reporter gene,
(iii) wherein a lower level of expression of the reporter gene in the cell contacted with the serum or fraction thereof relative to a cell that was not contacted with the serum or fraction thereof indicates that the serum or fraction thereof comprises an agent that decreases the expression of a sirtuin gene.

30. The method of claim 29, wherein the transcriptional control region is a promoter region.

31. The method of claim 29, wherein the sirtuin gene encodes SIRT1.

32. The method of claim 29, wherein the sirtuin gene encodes sir2.

33. A method for determining whether a subject is calorically-restricted or resistant to a stress condition, comprising determining the protein level of a factor in the subject, wherein a higher level of the factor in the subject relative to that of a control subject indicates that the subject is calorically-restricted or resistant to stress.

34. The method of claim 33, comprising determining the level of a sirtuin protein in a tissue of the subject.

35. The method of claim 34, wherein the sirtuin is SIRT1.

36. The method of claim 33, comprising determining the level of a factor in the serum of the subject, wherein the factor increases the level of expression of a sirtuin.

37. The method of claim 36, wherein the sirtuin is SIRT1.

38. A composition comprising serum from a calorically-restricted organism and a 293T or human embryonic kidney cell.

39. A composition comprising serum from a calorically-restricted organism that is not a rat or a monkey and a cell.

40. The composition of claim 39, wherein the cell is a mammalian cell.

41. The composition of claim 40, wherein the cell is a human cell.

42. The composition of claim 39, wherein the organism is a mammal.

43. The composition of claim 42, wherein the mammal is a human.

44. A composition comprising isolated serum from a non-calorically-restricted organism that is not a rat or a monkey and a cell.
